(19) United States
(12) Patent Application Publication
(10) Pub. No.: US 2018/0217120 A1
(43) Pub. Date: Aug. 2, 2018

(54) APPARATUS AND METHOD FOR MONITORING FUEL OIL DELIVERY

(71) Applicant: GREG HABER, Woodbury, NY (US)

(72) Inventor: GREG HABER, Woodbury, NY (US)

(21) Appl. No.: 15/915,153

(22) Filed: Mar. 8, 2018

Related U.S. Application Data
(63) Continuation-in-part of application No. 15/040,531, filed on Feb. 10, 2016, now Pat. No. 9,927,306.
Application No. 15/040,531 is a continuation-in-part of application No. 14/665,507, filed on Mar. 23, 2015, now Pat. No. 9,897,474.
(60) Provisional application No. 62/628,003, filed on Feb. 8, 2018.
(60) Provisional application No. 61/970,156, filed on Mar. 25, 2014.

(51) Int. Cl. G01N 33/28; G01F 1/115; G01F 1/075; G01N 11/04; G01F 15/02; G01N 21/3577; G01N 27/22

(52) U.S. Cl. CPC G01N 33/2841; G01F 1/1155; G01F 1/0755; G01N 27/221; G01F 15/022; G01N 21/3577; G01N 11/04

(57) ABSTRACT

The delivery of fuel oil through a fuel oil delivery pipe is monitored to measure the flow rate, temperature, viscosity, density and dielectric constant of the fuel oil as it moves through the delivery pipe. The digital data signals from the sensors which are a function of the measured parameters are recorded in a memory. An IR sensor detects the presence of air in the pipe and prevents the data signals from being recorded. The actual total quantity of fuel oil delivered through the pipe is calculated based upon the recorded data signals, which may be adjusted to take into account the temperature of the fuel oil being delivered. A clock circuit generates a timing signal reflecting the date and time the measurements were taken. Information as to the quantity delivered and the time of delivery may be sent to a remote location.

### CROSS-REFERENCE TO RELATED APPLICATIONS

This application claims priority on Provisional Patent Application Ser. No. 62/628003, filed Feb. 8, 2018, and is a continuation-in-part of U.S. patent application Ser. No. 15/040,531, filed Oct. 23, 2017, which is a continuation-in-part of U.S. patent application Ser. No. 14/665,507, filed Mar. 23, 2015, which claims priority on Provisional Patent Application Ser. No. 61/970156, filed Mar. 25, 2014, now U.S. Pat. No. 9,897,474, issued Feb. 20, 2018, all of which are incorporated herein in their entirety by reference.

### STATEMENT REGARDING FEDERALLY SPONSORED RESEARCH OR DEVELOPMENT

Not Applicable

### REFERENCE TO A “SEQUENCE LISTING”, A TABLE, OR A COMPUTER PROGRAM LISTING APPENDIX SUBMITTED ON COMPACT DISC

Not Applicable

### BACKGROUND OF THE INVENTION

### 1. Field of the Invention

The present invention relates to fuel oil delivery and more particularly to an apparatus and method for accurately monitoring, calculating and transmitting the actual quantity and quality of fuel oil delivered through a delivery pipe to a recipient, such as a residential or commercial building in which the amount present in the delivery is measured and taken into account in determining the actual quantity of fuel oil.

### 2. Description of Prior Art Including Information Disclosed Under 37 Cfr 1.97 and 1.98

Fuel oil delivery records provided by the delivery company reflecting the quantity of fuel oil delivered to a recipient are not always accurate because the pumps used on fuel oil delivery trucks are not always accurate. Given the current high cost of fuel oil for heating residential and commercial buildings and for other purposes, such inaccuracies can be very costly to the home owner or business proprietor, particularly if they occur repeatedly over time.

The pumps on the fuel oil delivery trucks can be inaccurate for several reasons. Aside from the fact that the meter associated with the pumps may not be correctly calibrated, the oil pressure through the meter is designed for a specific flow rate and fluctuations in the flow rate may cause an inaccurate reading of the quantity of oil delivered. The temperature of the oil is also a factor because it affects the volume of the oil. Heat will cause fuel oil to expand and hence occupy more volume than the same amount of fuel oil at a lower temperature. Thus, the quantity of fuel oil recorded as delivered may be inflated if the temperature of the fuel oil is higher at the time it is delivered.

The viscosity of the oil being delivered is an indication of the purity of the oil. Impure oil will have a different viscosity than impure oil.

The dielectric property of the oil being delivered is also a function of the purity of the oil.

In addition, the density of the oil being delivered indicates whether and how much air is present in the oil. The more air that is present in the oil, the more the volume of the oil will increase, further distorting the accuracy of the delivery records.

Accordingly, there is a need for an apparatus and method which can accurately monitor and record the actual quantity and quality of fuel oil delivered to residential and commercial buildings by taking into account fluctuations in flow rate and temperature of the fuel oil at the time of delivery as well as the viscosity, density and dielectric property of the oil.

The components of the apparatus require power to operate. External power supplies providing 120 volt alternate current can create a danger of explosion when used in conjunction with fuel oil. Accordingly, there is a need for providing sufficient power to the apparatus in a manner which eliminates this danger.

Another problem inherent in conventional fuel oil delivery monitoring systems is that air may be present in the delivery pipe but the mechanism used to monitory the amount of fuel oil passing through the pipe cannot tell the difference between fuel oil and air. Therefore air in the delivery pipe is counted as fuel oil, resulting in an inaccurate measure of the quantity of fuel oil actually delivered.

It is therefore a primary object of the present invention to provide an apparatus and method for accurately monitoring and recording the quantity and quality of fuel oil delivered to a residential or commercial building.

It is another object of the present invention to provide an apparatus and method for accurately monitoring and recording the quantity of fuel oil delivered to a residential or commercial building, which takes into account the temperature of the fuel oil at the time the fuel oil is delivered.

It is another object of the present invention to provide an apparatus and method for accurately monitoring and recording the quantity of fuel oil delivered to a residential or commercial building, which takes into account fluctuations in the flow rate of the fuel oil at the time the fuel is delivered.

It is another object of the present invention to provide an apparatus and method for accurately monitoring and recording the quantity of fuel oil delivered to a residential or commercial building, which takes into account the density of the fuel oil as the fuel oil is being delivered.

It is another object of the present invention to provide an apparatus and method for accurately monitoring and recording the quality of fuel oil delivered to a residential or commercial building, which takes into account the viscosity of the fuel oil as the fuel oil is being delivered.

It is another object of the present invention to provide an apparatus and method for accurately monitoring and recording the quality of fuel oil delivered to a residential or commercial building, which takes into account the dielectric property of the fuel oil as the fuel oil is being delivered.

It is another object of the present invention to provide an apparatus and method for accurately monitoring and recording the amount of fuel oil delivered to a residential or commercial building which measures the volume of the fuel oil as the fuel oil is being delivered.

It is another object of the present invention to provide an apparatus and method for accurately monitoring and recording the quantity of fuel oil delivered to a residential or commercial building which uses the temperature, flow rate, density and volume of the fuel oil measured at the time the fuel oil is delivered to calculate the actual total quantity of fuel oil delivered.

It is another object of the present invention to provide an apparatus and method for accurately monitoring and recording the quantity of fuel oil delivered to a residential or commercial building which is capable of calculating and displaying the actual total quantity of fuel oil delivered and the date and time the fuel oil was delivered.

It is another object of the present invention to provide an apparatus and method for accurately monitoring and recording the quantity of fuel oil delivered to a residential or commercial building which is capable of generating an audible alarm if the temperature of the fuel oil being delivered exceeds a pre-set level.

It is another object of the present invention to provide an apparatus and method for accurately monitoring and recording the quantity of fuel oil delivered to a residential or commercial building which is capable of calculating and transmitting to a remote location the actual total quantity of fuel oil delivered and the date and time the fuel oil was delivered.

It is another object of the present invention to provide an apparatus and method for accurately monitoring and recording the quantity of fuel oil delivered to a residential or commercial building which is capable of calculating and transmitting to a remote location the actual total quantity of fuel oil delivered and the date and time the fuel oil was delivered by WiFi or mode.

It is another object of the present invention to provide an apparatus and method for accurately monitoring and recording the quantity of fuel oil delivered to a residential or commercial building which includes a generator driven by the fuel oil flow for providing low voltage to power the components of the apparatus.

It is another object of the present invention to provide an apparatus and method for accurately monitoring and recording the quaintly of fuel oil delivered to a residential or commercial building which is capable of determining the difference between fuel oil and air in the delivery pipe and take into account only the fuel oil passing through the delivery pipe, resulting in an accurate count of the fuel oil being delivered.

### BRIEF SUMMARY OF THE INVENTION

To those and to other objects which may hereinafter appear, one aspect of the present invention relates to apparatus for monitoring and recording the delivery of fuel oil through a fuel oil delivery pipe including means associated with the oil delivery pipe for measuring the temperature, flow rate, viscosity, density and dielectric property of the fuel oil as it moves through the pipe. The apparatus includes means for generating data signals with are a function of the measured parameters. Means are also provided for recording the data signals and for calculating the actual total quantity of fuel oil delivered through the pipe based upon the data signals.

Means are provided for recording the time that the temperature and flow rate parameters were measured and for generating a time signal which is a function thereof.

Means are provided for generating a transmission signal which is a function of the calculated actual total quantity of fuel oil delivered and the time signal.

Means are also provided for transmitting the transmission signal to a remote location. The transmitting means may include a WiFi transmitter or a modem for transmitting the transmission signals through the internet.

Means are also provided for generating an audible alarm if the temperature of the fuel oil being delivered exceeds a pre-set level.

Means are also provided for generating low voltage to power the apparatus components which is driven by the flow of fuel oil.

In accordance with another aspect of the present invention, a method is provided for monitoring the delivery of fuel oil through a fuel oil delivery pipe, the method includes the steps of measuring the temperature, flow rate, viscosity, density and dielectric property of the fuel oil as it moves through the delivery pipe, generating data signals which are a function of the measured parameters of the fuel oil, recording the data signals, and calculating the actual total quantity of fuel oil delivered through the pipe based upon the data signals.

The method includes the step of displaying the calculated actual total quantity of fuel oil delivered through the pipe.

The method includes the step of generating and displaying a time signal indicating the date and time that the temperature and flow rate of the fuel oil were measured.

The method further includes generating a transmission signal which is a function of the calculated actual total quantity of fuel oil delivered and time signal.

The method further includes transmitting the transmission signal to a remote location using a WiFi transmitter or sending the transmission signal to a remote location through the internet using a modem.

The method further includes generating an audible alarm if the temperature of the fuel oil being delivered exceeds a pre-set level.

The method further comprises generating low voltage to power the apparatus components using a generator driven by the flow of fuel oil.

In accordance with another aspect of the present invention, apparatus for monitoring the delivery of fluid including fuel oil and air through a fuel oil delivery pipe is provided. The apparatus includes a flow meter associated with the fuel oil delivery pipe including a rotatable part for measuring the flow rate of fluid as it moves through the pipe. The flow meter generates data signals with are a function of the measured flow rate of the fluid as it moves through the delivery pipe. A sensor detects air as it moves through the delivery pipe, The sensor generates an output signal when air is detected. Means are provided for recording the data signals from the flow meter when the output signal from the air sensor is not present. Means are also provided for calculating the actual total quantity of fuel oil delivered through the pipe based upon the recorded data signals.

The sensor preferably includes an infrared sensor. The means are provided for measuring the properties of the fuel oil. Those means include means for measuring the temperature of the fuel oil as it is delivered and for adjusting the recorded data signal in accordance with the difference between the measured temperature and the known volume of fuel oil at a given temperature.

The recorded data signal is multiplied by an oil expansion factor to adjust for the difference in volume between the volume of fuel oil at the measured temperature of the fuel oil passing through the pipe and the volume of fuel oil at a given temperature.

The apparatus also includes means for measuring the viscosity of the fuel oil, means for measuring the density of the fuel oil and means for measuring the dielectric constant of the fuel oil.

The apparatus further includes means for recording the calculated actual total quantity of fuel oil delivered through the pipe.

The apparatus further includes a low voltage electric generator driven by movement of fuel oil through the fuel oil delivery pipe to supply power to the apparatus components.

In accordance with another aspect of the present invention, a method is provided for monitoring the delivery of fluid including fuel oil and air through a fuel oil delivery pipe. The method includes the steps of measuring the flow rate of the fluid as it moves through the delivery pipe using a flow meter with a rotatable part. Data signals are generated witch are a function of the measured flow rate based upon the rotation of the rotational part of the flow meter. A determination is made as to when air is present in the delivery pipe. An output signal is generated when air is detected. The data signals from the flow meter are recorded when the output signal from the air detector is not present. The actual total quantity of fuel oil delivered through the pipe is calculated based upon the recorded data signals.

The step of determining when air is present is performed by an infrared sensor.

The method further includes the step of measuring the temperature the fuel oil as it is delivered and adjusting the recorded data signal in accordance with the difference between the measured temperature and the known volume of fuel oil at a given temperature.

The step of adjusting the recorded data signal is performed by multiplying the measured temperature by an oil expansion factor to adjust for the difference in volume between the volume of fuel oil at the measured temperature of the fuel oil passing through the pipe and the volume of fuel oil at a given temperature.

The method further includes the step of measuring the viscosity of the fuel oil, the step of measuring the density of the fuel oil, and the step of measuring the dielectric constant of the fuel oil.

The method further includes the step of generating a low voltage electric signal to power the components driven by movement of fuel oil through the fuel oil delivery pipe.

### BRIEF DESCRIPTION OF THE SEVERAL VIEWS OF DRAWINGS

To these and to such other objects that may hereinafter appears, the present invention relates to an apparatus and method for monitoring fuel oil delivery as described in detail in the following specification and recited in the annexed claims, taken together with the accompanying drawings, in which like numerals refer to like parts and in which:

FIG. 1 is a block diagram of the apparatus of the first preferred embodiment of the present invention;

FIG. 2 is a schematic diagram of the circuit of the first preferred embodiment of the present invention;

FIG. 3 is a flow chart of the method of the first preferred embodiment of the present invention;

FIG. 4 is a block diagram of the apparatus of the second preferred embodiment of the present invention; and

FIG. 5 is a flow chart of the method of the second preferred embodiment of the present invention.

### DETAILED DESCRIPTION OF THE INVENTION

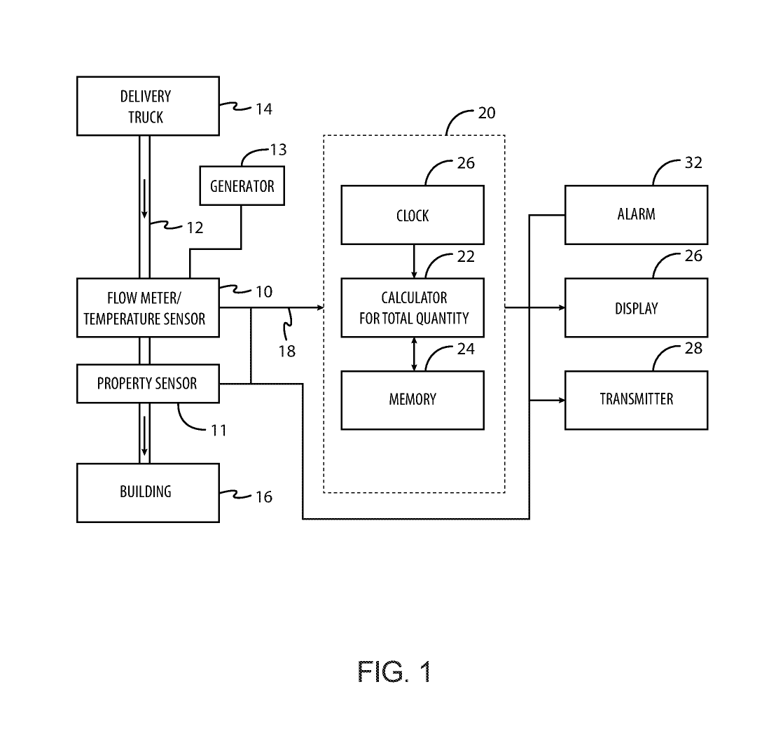

As seen in FIG. 1, which shows the first preferred embodiment of the present invention, the flow meter component 10 and the property sensor 11 are inserted into the delivery pipe 12 which extends between the fuel oil delivery truck 14 and the building 16 to which the fuel oil is to be delivered. Truck 14 has an associated pump which causes the fuel oil in the truck tank to flow through pipe 12 to a fuel storage tank within or proximate to building 16. Pipe 12 is customarily at least 2 inches in diameter such that the fuel oil can be transferred from the truck to the building in a relatively short time.

Flow meter component 10 may include a fuel or oil flow meter designed for use with a 2 inch pipe, such as Part No. 113900-9502, commercially available from Great Plains Industries of Santa Ana, Calif. The flow meter includes an internal wheel situated in the flow path which revolves. The number of revolutions per time interval provides a measurement of the flow rate of the fuel oil passing through pipe 12. The flow meter generates a digital data flow signal which is a function of the measured flow rate of the fuel oil as it passes through the meter.

A low voltage electric generator 13 is mechanically connected to flow meter 10 such that the rotation of the internal wheel of the flow meter drives the generator to provide a low voltage to power the components of the apparatus. Avoiding the use of a high voltage external source to power the apparatus components eliminates the danger of explosion resulting from a spark igniting fuel oil fumes.

Property sensor 11 includes a temperature sensor and a density measurement gauge. Property sensor 11 also includes a means for measuring the viscosity of the fuel oil and a detector for measuring the electrical properties of the fuel oil to determine the dielectric constant of the oil.

The property sensor 11 includes electronics which convert the sensed temperature and density measurement into a digital temperature data quantity signal and a digital density data quantity signal. The viscosity measurement and dielectric measurement are converted into digital viscosity and dielectric data purity signals.

The property sensor may take the form of Fluid Property Sensor FPS2800B12C4 available from Measurement Specialties, Inc. of 105 ay. du General Eisenhower BP 23705 31037 TOULOUSE CEDEX 1, France or the equivalent. That device is capable of directly and simultaneously measuring the viscosity, density, dielectric constant and temperature of the fuel oil as it flows through the sensor. The sensor monitors the direct and dynamic relationship between multiple physical properties to determine the quality, condition and contaminant loading of fluids such as fuel oil. It is a fully integrated, stand-alone module which combines sensor and processing electronics, including an on-board microprocessor for real-time data analysis.

The data flow signals from component 10 and the data quantity signals from property sensor 11 are transferred to the electronic circuit 20 of the apparatus through a wire or cable 18. Circuit 20 includes a calculator circuit 22, an electronic memory 24 and a clock or timer circuit 26. Circuit 20 calculates the actual total quantity of fuel oil which is delivered to the building taking into account the flow rate fluctuations, and the temperature and density measurements reflected in the data signals. The actual total quantity of fuel oil delivered may be displayed in numbers of gallons or liters.

The viscosity and dielectric data purity signals from property sensor 11 are compared to pre-determined values of viscosity and dielectric constant. If either or both of those data impurity signals deviate from pre-determined values by a given amount, a data impurity signal is generated on a wire or cable to display 26.

A data impurity signal would indicate that the fuel oil being delivered is not of sufficient quality. For example such an event could occur if the fuel oil has been mixed with used oil. That signal could also actuate alarm 32 to provide an alert that the fuel being delivered is of sub-standard quality.

Circuit 20 also generates a time signal which represents the date and time that the fuel oil was delivered to the building. The time signal is based upon the output of an internal clock or timer circuit.

The data signals reflecting flow rate, temperature and density, the calculated actual total quantity delivered and the time signal are stored in memory 24. Some or all of those signals can also be displayed on an LCD display 26 so that they can be observed at the site of apparatus and/or sent to a transmitter 28 for forwarding to a remote location for display and/or recording.

The data impurity signal on wire or cable 19 is transferred directly an alarm 32, display 26 and transmitter 28 to alert the recipient of the fuel oil immediately that there may be an issue with the purity of the fuel oil being delivered.

The apparatus can be programmed to display and/or transmit various parameters including current flow rate in gallons (or liters), the temperature of the fuel oil, the type of fuel oil (gasoline or diesel) and the calculated actual total quantity of fuel oil delivered in gallons (or liters).

Transmitter 28 could take the form a WiFi transmitter for wireless communication. It could also take the form of an internet-connected computer with a modem for communication over the internet.

An alarm circuit 32 connected to property sensor 11 and circuit 20 is actuated to generate an audible alarm signal when the quality of the fuel oil being delivered is unacceptable or the temperature sensed by the temperature sensor exceeds a pre-set level. The level at which the alarm will be actuated can be adjusted. The alarm will provide a real time notification if the quality of the fuel oil being delivered is below a pre-set standard or the temperature of the fuel oil being delivered is too high. In indication that the alarm has been actuated will be stored in memory 24.

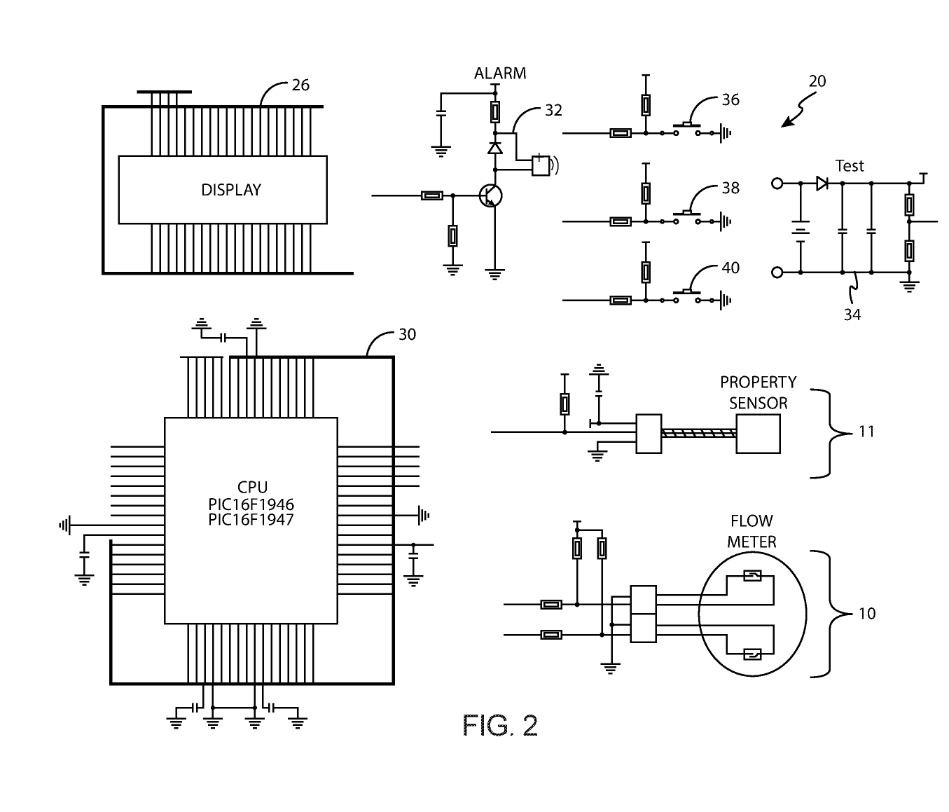

FIG. 2 illustrates some of the basic sub-circuits which form circuit 20 and are connected to receive the output of the flow meter 10 and property sensor 11. Those sub-circuits include a LCD display 26 and a CPU 30 which controls the overall function of the apparatus and forms the calculated actual total quantity of fuel oil delivered based on the data signals.

CPU 30 may be a 64-pin, flash based, 8 bit CMOS microcontroller with a LCD driver such as is commercially available from Microchip Technology Inc. of Chandler, Ariz. as Part no. PIC16f1946/PIC16f1947.

The calculated actual total quantity of fuel oil delivered is formed in the CPU by multiplying the coefficient of cubical or thermal expansion of the particular fuel being delivered per temperature degree times a factor based up the number of revolutions of the internal wheel of the flow meter per time resulting from the fluid flow. For example, the expansion factor for diesel fuel is 0.0008/c degree and the flow rate factor is 0.0747 L for a pipe of 2 inch diameter.

Memory 24 is programmed to store the expansion factors for a variety of common liquid fuels. The type of fuel being delivered is also entered into the apparatus. Circuit 20 uses that information and the measured temperature of the fuel at the time it is delivered to form the calculated actual total quantity of the fuel delivered.

An output of CPU 30 is connected to a driver circuitry for a alarm 32 which creates an audible signal if the quality is unacceptable or the sensed temperature exceeds a given level. The level at which the alarm is actuated can be adjusted. An indication that the alarm has been actuated is stored in memory 24. A test circuit 34 and three programming switches 36, 38 and 40 are also connected to CPU 30.

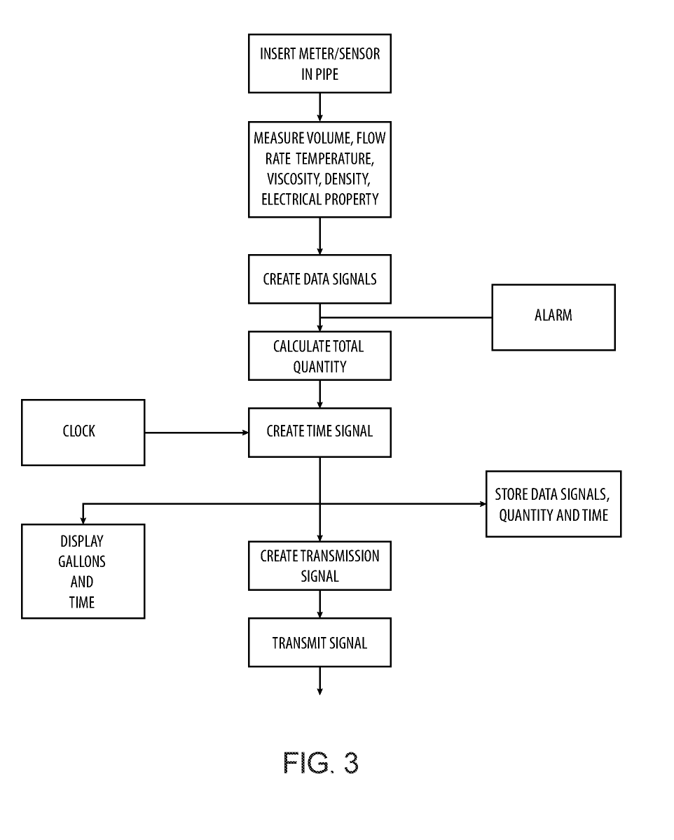

FIG. 3 is a flow chart of the steps of the method of the first preferred embodiment of the present invention. After the flow meter and property sensor are inserted into the delivery pipe and circuit is initialized, the flow rate, temperature, viscosity, density and dielectric constant of the fuel oil passing through the pipe are measured and the data signals are created. The CPU receives the data quantity signals and calculates the actual total quantity of fuel oil delivered in gallons or liters. The time signal is created in accordance with the output of the clock to reflect the date and time at which the parameters were measured.

The calculated actual total quantity delivered and time signal are displayed by the LCD display and stored in the memory. A transmission signal may be created based on the calculated actual total quantity delivered and the time signal. The transmission signal may then be sent to a remote location, if desired. The transmission may be accomplish using a wireless connection, such a WiFi, or through the internet using an internet-connected computer and a modem.

In the event that the quality of the fuel oil is unacceptable or the temperature which exceeds a pre-set level is sensed, an alarm is actuated to generate an audible signal indicating an unacceptable quality or a high temperature condition. The actuation of the alarm is stored in memory for future reference.

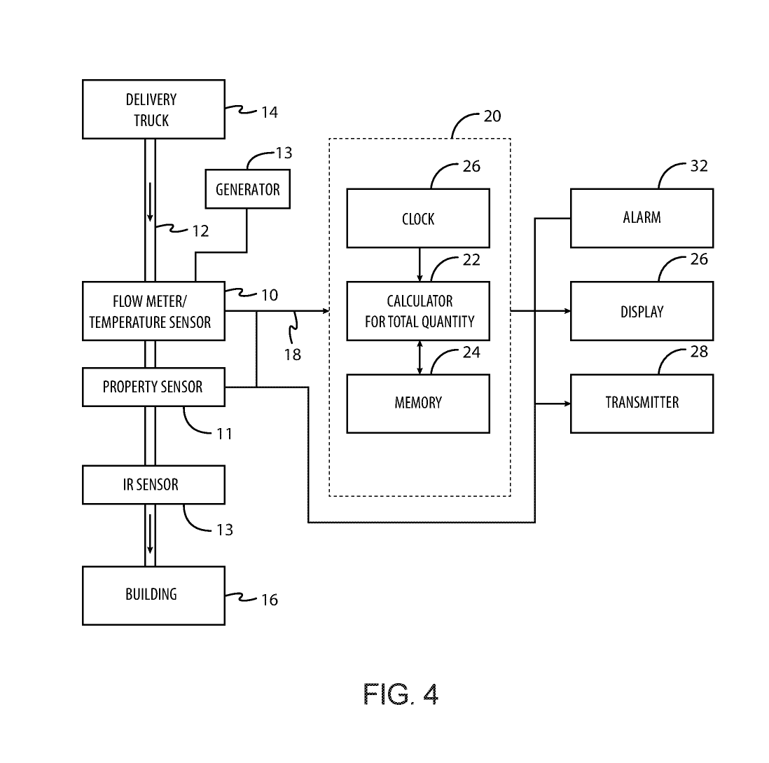

FIG. 4 is a block diagram of the second preferred embodiment of the present invention. The second preferred embodiment of the present invention includes the same components as the first preferred embodiment except for Infrared (IR) Sensor 13 which is associated with delivery pipe 12 and detects where the fluid passing through the delivery pipe 12 to building 16 at any particular time during delivery is air. If Sensor 13 detects air in the pipe, it generates an output signal through cable 18 to circuit 20 and in particular calculator 22.

Data signals from flow meter 10 are provided to calculator 22 through wire or cable 18. Those data signals are generated by the flow meter which includes a rotatable wheel which is rotated by the fluid in the delivery pipe flows past the meter. The data signals reflect the flow rate of the fluid as it passes the meter. Magnets are associated with or mounted on the meter wheel. As the wheel is rotated, a Hall effect sensor picks up the magnetic field created by the moving magnets and generates the data signals as a function of the wheel rotation.

The data signals received by calculator 22 are normally recorded in memory 24. However, if the IR Sensor 13 is generating an ouput signal, indicating that it is air, not fuel oil, that is being delivered through the pipe, the data signals are blocked from being recorded in the memory and will not be used as part of the calculation of the total fuel oil delivered. This system insures that the total fuel oil delivered with not include a false reading attributable to the air in the delivery pipe. Preferably, display 26 will display the number of gallons of fuel oil delivered and the amount of air detected by the IR Sensor in the delivery pipe.

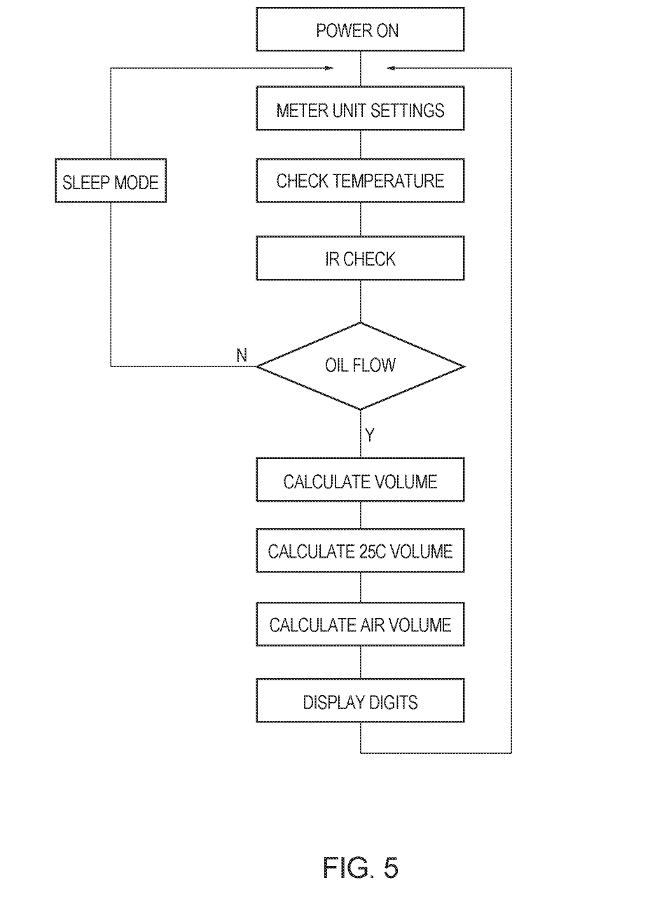

FIG. 5 is a flow chart of the second preferred embodiment of the present invention in which the amount of air in the delivery pipe is measured and displayed so that the quantity of air in the fluid in the delivery pipe can be taken into account in calculating the actual quantity of fuel oil delivered. In this embodiment, an infrared sensor 13 can be situated anywhere along the delivery pipe 12 although in FIG. 4, the sensor is shown as situated between sensors 10, 11 and building 16. However, in certain situations, the IR Sensor could be substituted for sensor 11, and connected to calculator 22 and display 26.

When the power is turned on, the flow meter settings are initiated, and the data signals indicating the flow rate, temperature and other properties of the fluid flowing through the delivery pipe are generated, as in the previous embodiment. In addition, the IR Sensor 13 senses whether air flowing though the delivery pipe. If air is detected, the IR Sensor generates an output signal which prevents the data signals from being recorded in the memory during the period in which the output signal from the sensor is being generated.

The volume of fuel oil is calculated as in the previous embodiment based upon the recorded data signals. The flow rate of the fuel oil through the delivery pipe as reflected in the recorded data signals is adjusted to compensate for the temperature of the fuel oil sensed by the temperature sensor because the volume of the fuel oil increases with temperature.

This adjustment takes place in calculator 22 which adjusts the sensed volume indicated by the recorded data signals to the known volume of fuel oil at a particular temperature, for example 25C. degrees, by multiplying the sensed volume by a factor dependent upon the type of fuel oil being delivered.

The total quantity of fuel oil, adjusted by the temperature and not including any air in the fluid being monitored, is then displayed on display 26. Although it is optional, the total amount of air sensed by the IR Sensor may also be displayed on the display, separately from the fuel quantity.

While only a limited number of preferred embodiments of the present invention have been disclosed for purposes of illustration, it is obvious that many modifications and variations could be made thereto. It is intended to cover all of those modifications and variations which fall within the scope of the present invention, as defined by the following claims.

I claim:

1. Apparatus for monitoring the delivery of fluid including fuel oil and air through a fuel oil delivery pipe comprising a flow meter associated with the fuel oil delivery pipe including a rotatable part for measuring the flow rate of fluid as it moves through the pipe, said flow meter generating data signals with are a function of the measured flow rate of the fluid as it moves through the delivery pipe, a sensor for detecting air as it moves through the delivery pipe, said sensor generating an output signal when air is detected, means for recording the data signals from said flow meter when said output signal from said sensor is not present and means for calculating the actual total quantity of fuel oil delivered through the pipe based upon the recorded data signals.

2. The apparatus of claim 1 wherein said sensor comprises an infrared sensor.

3. The apparatus of claim 1 wherein said means for measuring the properties of the fuel oil comprises means for measuring the temperature of the fuel oil as it is delivered and for adjusting the recorded data signal in accordance with the difference between the measured temperature and the known volume of fuel oil at a given temperature.

4. The apparatus of claim 3 wherein said recorded data signal is multiplied by an oil volume expansion factor to adjust for the difference in volume between the volume of fuel oil at the measured temperature of the fuel oil passing through the pipe and the volume of fuel oil at a given temperature.

5. The apparatus of claim 1 further comprising means for measuring the viscosity of the fuel oil.

6. The apparatus of claim 1 further comprises means for measuring the density of the fuel oil.

7. The apparatus of claim 1 further comprising means for measuring the dielectric constant of the fuel oil.

8. The apparatus of claim 1 further comprising a low voltage electric generator driven by movement of fuel oil through the fuel oil delivery pipe to supply power to the apparatus components.

9. A method for monitoring the delivery of fluid including fuel oil and air through a fuel oil delivery pipe, the method comprising the steps of:
(a) measuring the flow rate of the fluid as it moves through the delivery pipe using a flow meter with a rotatable part;
(b) generating data signals with are a function of the measured flow rate based upon the rotation of the rotational part of the flow meter;
(c) determining when air is present in the delivery pipe and generating an output signal when air is detected;
(d) recording the data signals from the flow meter when the output signal is not present; and
(e) calculating the actual total quantity of fuel oil delivered through the pipe based upon the recorded data signals.

10. The method of claim 9 further comprising the step of determining when air is present is performed by an infrared sensor.

11. The method of claim 9 further comprising the step of measuring the temperature the fuel oil as it is delivered and for adjusting the recorded data signal in accordance with the difference between the measured temperature and the known volume of fuel oil at a given temperature.

12. The method of claim 9 further comprising the step of adjusting the recorded data signal is performed by multiplying the measured temperature by an oil expansion factor to adjust for the difference in volume between the volume of fuel oil at the measured temperature of the fuel oil passing through the pipe and the volume of fuel oil at a given temperature.

13. The method of claim 9 further comprising the step of measuring the viscosity of the fuel oil.

14. The method of claim 9 further comprising the step of measuring the density of the fuel oil.

15. The method of claim 9 further comprising the step of measuring the dielectric constant of the fuel oil.

16. The method of claim 9 further comprising the step of generating a low voltage electric signal to power the components driven by movement of fuel oil through the fuel oil delivery pipe.
